(19) United States
(12) Patent Application Publication
(10) Pub. No.: US 2026/0315806 A1
(43) Pub. Date: Oct. 15, 2026

(54) SYSTEMS AND METHODS FOR CLOSED-LOOP CONTROL OF PERIPHERAL NERVES

(71) Applicant: Georgia Tech Research Corporation, Atlanta, GA (US)

(72) Inventors: Alex Abramson, Atlanta, GA (US); Ramy Ghanim, Atlanta, GA (US); Yoon Jae Lee, Atlanta, GA (US); Woon Hong Yeo, Atlanta, GA (US)

(21) Appl. No.: 19/571,966

(22) Filed: Mar. 19, 2026

Related U.S. Application Data
(60) Provisional application No. 63/774,299, filed on Mar. 19, 2025.

(51) Int. Cl. A61N 1/36; A61N 1/04; A61N 1/05; A61N 1/372; A61N 1/378

(52) U.S. Cl. CPC A61N 1/36139; A61N 1/0476; A61N 1/0556; A61N 1/36031; A61N 1/36125; A61N 1/37205; A61N 1/378

(57) ABSTRACT

The present disclosure relates to a wearable printed circuit board (PCB), an implantable printed circuit board (PCB), and a communication method for transmitting signals between the two modules.

### RELATED APPLICATIONS

This application claims the benefit of priority to U.S. Provisional Application No. 63/774,299, filed Mar. 19, 2025.

### GOVERNMENT SUPPORT

This invention was made with government support under grant number GM150689, awarded by the National Institutes of Health. The government has certain rights in the invention.

### BACKGROUND

Ingestible and implantable bioelectronic therapeutics have the capability to function in closed loop therapies but are often constrained by communication protocols. Typical closed-loop therapies, namely the insulin hybrid closed-loop, utilize Bluetooth low energy (BLE) system on chips (SoCs) or radio frequency (RF) antennas to transmit and receive these signals. However, these components are associated with significant power consumption and a locational dependence between communicating devices. Additionally, these components often increase the physical size of the bioelectronic device making them incompatible for some anatomical locations. Here we demonstrate proof of the concept of a closed loop neurostimulator with the capability of autonomously stimulating a peripheral nerve in response to movement detection by a wearable sensor via in-body communication. We demonstrate in vivo efficacy of simultaneous bilateral closed-loop nerve stimulation in a small animal via utilization of wearable and implantable bioelectronics.

Body-conformal sensors and tissue-interfacing robotic therapeutics enable the real-time monitoring and treatment of diabetes, wound healing, and other critical conditions. By integrating sensors and drug-delivery devices, scientists and engineers have developed closed-loop drug-delivery systems with on-demand therapeutic capabilities to provide just-in-time treatments that correspond to chemical, electrical, and physical signals of a target morbidity. One of the most significant challenges is enabling these devices to communicate without exceeding device power and space constraints. A communication system entails hardware including supporting electronics and a medium for relaying signals. A closed-loop system requires a sensor, communication system, and an actuator.

Despite the demonstration of dozens of bioelectronic closed-loop therapeutic devices in small and large animal models, very few have made it into the clinic. Some have employed BLE which (1) suffers from heavy power consumption, decreasing device lifetime and (2) poor deep tissue transmission due to scattering effects. Others have used RF antennas which are less space and energy intensive—but require large external antennas and spatial alignment between transmitting and receiving antennas. These communication protocols are not adequate for developing closed-loop therapeutic systems that integrate body-worn sensors with internalized therapeutic devices such as neurostimulators and drug delivery devices. For these and other reasons, needs exist for improved in-body communication systems, devices, and methods. In our invention summary, we detail how our closed-loop system increases device lifetimes, minimizes space dedicated for hardware and supporting electronics, and decreases the need for spatial alignment between communicating devices.

### SUMMARY OF THE INVENTION

In certain aspects, the present disclosure provides a medical device system, comprising: a first electronic device in contact with tissue; a second electronic device in contact with tissue; and a pad; wherein: the first electronic device is placed outside of the body; the second electronic device is placed inside of the body; the first electronic device is configured to transmit an electrical signal through the tissue to the second electronic device, wherein a voltage gradient within the body is generated; and the pad is configured to harvest the generated voltage gradient, wherein the pad is placed at a predetermined distance within the body.

In certain aspects, the present disclosure provides for passage of an electrical signal from an electronic device touching tissue to another device touching tissue through the tissue in order to generate a voltage gradient in the body that can be harvested by a pad placed at given distance in the body.

In certain aspects, the present disclosure provides a wearable printed circuit board (PCB) comprising: a multi-layer and/or flexible substrate carrying a microcontroller unit (MCU), a power regulation stage with a DC-DC converter supplying 1-24 V for stimulation pulses, one or more output drivers for pulse generation, and/or a Bluetooth Low Energy (BLE) radio transceiver or equivalent wireless module for programming and data logging; wherein: the output driver(s) is/are selected from transistor(s), MOSFET(s), and power switch(es); and the wearable printed circuit board is configured to measure signals from at least one sensor, process the signals using the integrated microcontroller unit and deliver an electrical stimulation pulse upon exceeding a predetermined threshold.

In certain aspects, the present disclosure provides methods of treating, preventing, or diagnosing a disease or disorder, comprising attaching to a subject in need thereof a device or wearable PCB of the disclosure.

### BRIEF DESCRIPTION OF THE DRAWINGS

FIG. 1 The exemplary architecture and component of the wearable PCB layout and its major functional blocks.

FIG. 2 In-body communication architecture. FIG. 2A A custom flexible printed circuit board (FPCB) continuously monitors a strain sensor and automatically emits a variable (1-12V) square wave pulse to trigger the implantable network. Bioelectronics can be switched individually or synergistically. FIG. 2B Block diagram for closed-loop control and monitoring of an implantable actuator via IBC. FIG. 2C Photo of wearable FPCB. FIG. 2D Photo of implantable FPCB with voltage harvesting pads and nerve actuator. FIG. 2E Cyclic testing of laser-induced graphene (LIG) strain sensor with threshold for wearable pulsing specified.

FIG. 3. In-body communication enables low-power networks of wearable and implantable therapeutics. A wearable hub outfitted with a pulse generator creates a voltage gradient across tissue. Upon detection of the voltage gradient, the implantable network changes the bioelectronics' on-off state. Feedback control is accomplished by integration of a physical sensor to the wearable hub.

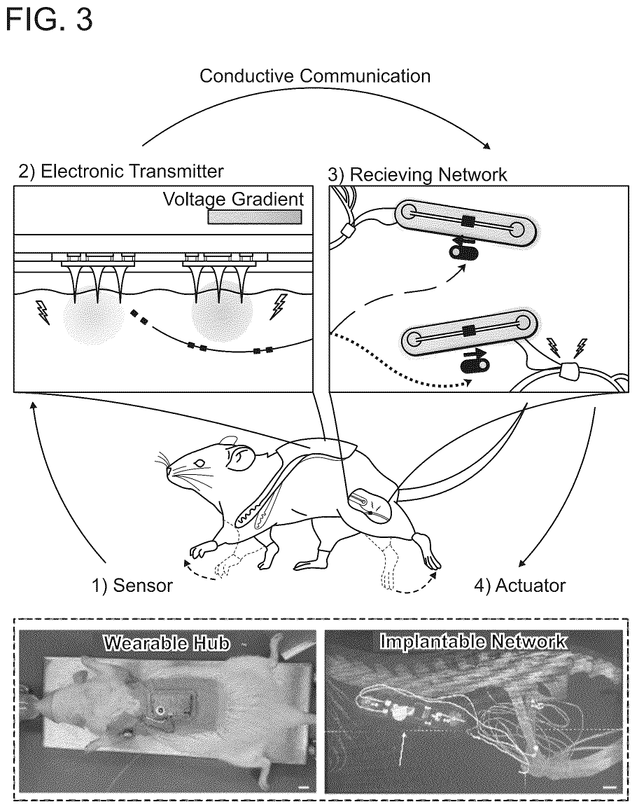

FIG. 4 Bio-integrated electronic networks for neurostimulation. FIG. 4A Schematic and image of rat outfitted with the described neurostimulator system. FIG. 4B Upon stimulus detection on either forelimb, the wearable board outputs either a 7V or 3V pulse. FIG. 4C resulting in triggering of either an NPN receiver or PNP receiver (n=30 over 3 rats, mean and SD plotted, **** indicates p<0.01 in unpaired t-test with Welch's correction). FIG. 4D Triggering of the receiver led to leg twitches (n=30 over 3 rats, mean and SD plotted, **** indicates p<0.01 in unpaired t-test with Welch's correction). FIG. 4E Representative image of motor function initiated by the wearable hub via IBC in the (translucent) resting and (opaque) stimulated state.

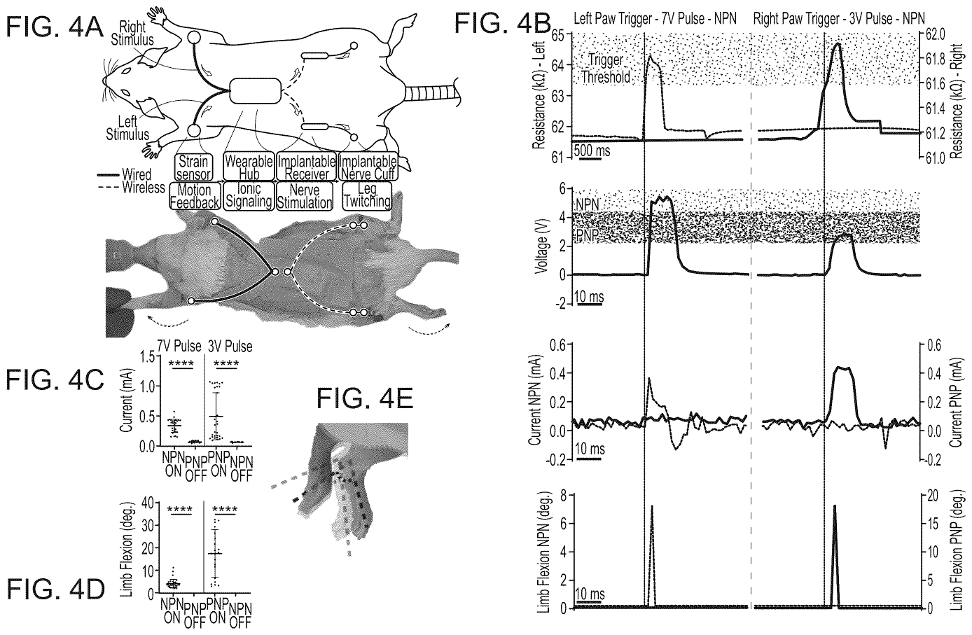

FIG. 5 BLE current consumption profiling and power consumption comparison across communication methods. FIG. 5A Representative BLE current profile measured using the nRF Profiler, illustrating average and peak current consumption during discrete operational stages: pre-processing, ramp-up, standby, radio startup, RX (receiving), radio switching, TX transmission, and post-processing. FIG. 5B Comparative summary of theoretical and experimental power consumption metrics (mean±SD, n=3) for Implantable transistor-based system, Bluetooth Low Energy (BLE), and Near Field Communication (NFC) communication. The table highlights standby currents, active currents, daily power consumption, and estimated battery lifetimes for each technology, demonstrating the substantial energy-saving advantages of the implantable transistor-based system compared to BLE and NFC.

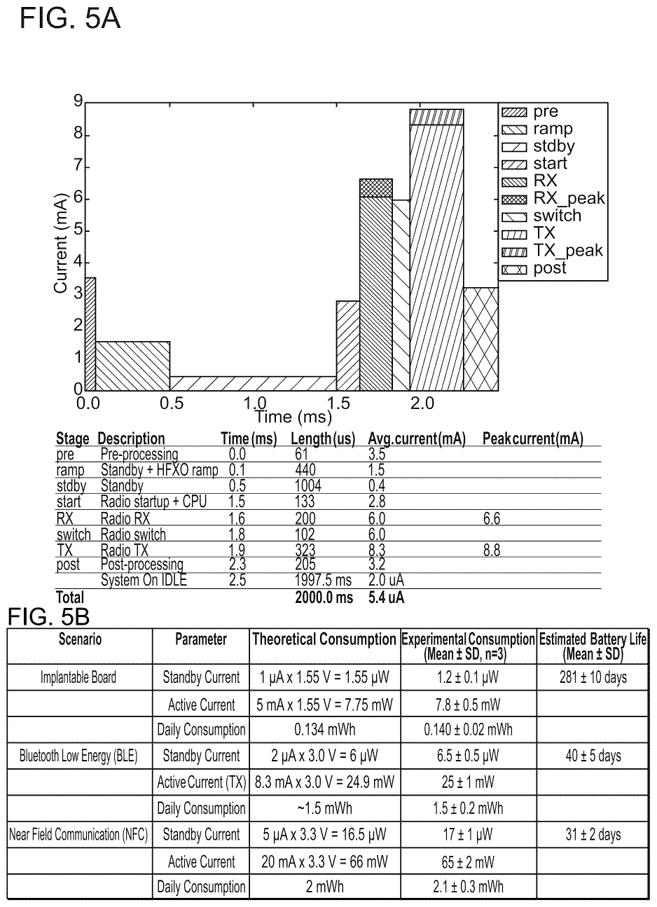

FIG. 6 Multiplexed wireless sensing utilizing serpentine traces and laser-induced graphene sensors. FIG. 6A Fabrication of a uniaxial laser-induced graphene (LIG) sensor. FIG. 6B Fabrication of bilayer copper-polyimide serpentine traces. FIG. 6C Strain-resistance calibration curve of 10 LIG sensors. FIG. 6D Strain-resistance calibration curve of 9 LIG sensors with Sensor 6 from (C) being removed. FIG. 6E Multiplexed sensing of forelimb motion using the wearable hub and LIG sensors.

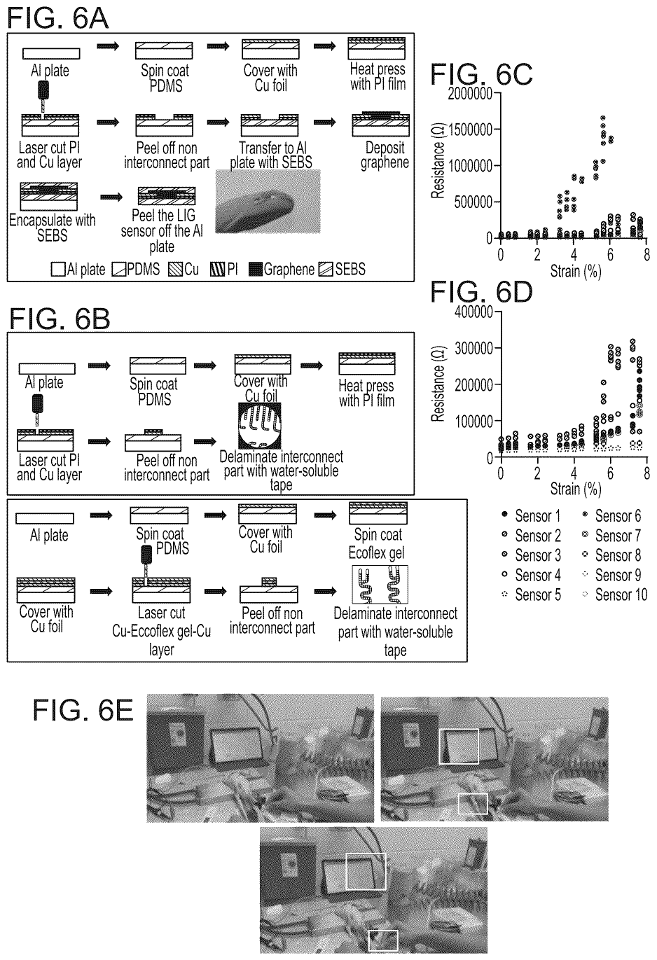

FIG. 7 Layout of example system within body communication receiver/harvesting pads, a coin cell battery, a transistor switch, a coin cell battery, and a location for actuator placement.

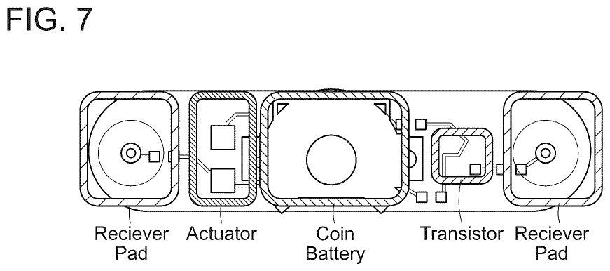

### DETAILED DESCRIPTION OF THE INVENTION

In certain aspects, the present disclosure provides a medical device system, comprising: a first electronic device in contact with tissue; a second electronic device in contact with tissue; and a pad; wherein: the first electronic device is placed outside of the body; the second electronic device is placed inside of the body; the first electronic device is configured to transmit an electrical signal through the tissue to the second electronic device, wherein a voltage gradient within the body is generated; and the pad is configured to harvest the generated voltage gradient, wherein the pad is placed at a predetermined distance within the body.

In certain aspects, the present disclosure provides for passage of an electrical signal from an electronic device touching tissue to another device touching tissue through the tissue in order to generate a voltage gradient in the body that can be harvested by a pad placed at given distance in the body.

In certain embodiments, the medical device comprises: a wearable printed circuit board (PCB); an implantable printed circuit board (PCB); and a harvesting pad; wherein: the wearable PCB is configured to transmit an electrical signal to the implantable PCB through the tissue, wherein a voltage gradient within the body is generated; and the harvesting pad is configured to harvest the generated voltage gradient, wherein the harvesting pad is placed at a predetermined distance within the body.

In certain embodiments, the device further comprises a transistor, wherein the transistor is switched on and off by the harvested voltage gradient.

In certain embodiments, the transistor is utilized to switch on a device, wherein the device is selected from a group consisting of a neurostimulator, a degradable metal film, a pump, a charger, a light emitting diode (LED), a triggerable auto-injector, a motor, a pneumatic inflater, an electrolyser, and a microcontroller; wherein the device is for the purposes selected from neurostimulation, drug delivery, collection of biosignals such as temperature, pH, and gas concentrations, oxygen generation to treat and monitor diabetes, wound healing, treatment of myocardial infarctions, treatment of epilepsy, and treatment of Parkinson's disease.

In certain embodiments, the device further comprises a sensor, wherein the sensor is placed on one location of an animal and is used to trigger a device in a separate location.

In certain embodiments, the device further comprises a strain sensor, wherein the strain sensor is placed on one location of an animal and is used to trigger a neurostimulation event via a nerve cuff in a separate location or the same location.

In certain embodiments, the first electronic device is a wearable printed circuit board (PCB), and the wearable printed circuit board (PCB) produces an electrical signal, wherein the electrical signal is an emitted pulse.

In certain embodiments, the device further comprises a sensor connected to the wearable PCB, wherein the sensor is a chemical or physical sensor selected from a group consisting of a glucose sensor, a physiological analyte, a strain sensor, a pressure sensor, an EEG sensor, and an EKG sensor; and wherein the wearable PCB can emit the pulse when the sensor reaches a threshold value.

In certain embodiments, the wearable PCB is configured to emit the pulse when a user inputs a command by touching the wearable PCB to emit the pulse. In further embodiments, the wearable PCB is configured to emit the pulse when a user inputs a command by using a smartphone app that sends a signal to the wearable PCB.

In certain embodiments, the wearable PCB is configured to emit the pulse at a predetermined or preprogrammed frequency and/or schedule wherein the frequency and/or schedule are selected from once every second, once every minute, once every hour, or once every day.

In certain embodiments, the frequency and/or schedule are selected from approximately once every second, once every 2 seconds, once every 3 seconds, once every 4 seconds, once every 5 seconds, once every 6 seconds, once every 7 seconds, once every 8 seconds, once every 9 seconds, once every 10 seconds, once every 11 seconds, once every 12 seconds, once every 13 seconds, once every 14 seconds, once every 15 seconds, once every 16 seconds, once every 17 seconds, once every 18 seconds, once every 19 seconds, once every 20 seconds, once every 21 seconds, once every 22 seconds, once every 23 seconds, once every 24 seconds, once every 25 seconds, once every 26 seconds, once every 27 seconds, once every 28 seconds, once every 29 seconds, once every 30 seconds, once every 31 seconds, once every 32 seconds, once every 33 seconds, once every 34 seconds, once every 35 seconds, once every 36 seconds, once every 37 seconds, once every 38 seconds, once every 39 seconds, once every 40 seconds, once every 41 seconds, once every 42 seconds, once every 43 seconds, once every 44 seconds, once every 45 seconds, once every 46 seconds, once every 47 seconds, once every 48 seconds, once every 49 seconds, once every 50 seconds, once every 51 seconds, once every 52 seconds, once every 53 seconds, once every 54 seconds, once every 55 seconds, once every 56 seconds, once every 57 seconds, once every 58 seconds, once every 59 seconds, and once every 60 seconds.

In certain embodiments, the frequency and/or schedule are selected from approximately once every minute, once every 2 minutes, once every 3 minutes, once every 4 minutes, once every 5 minutes, once every 6 minutes, once every 7 minutes, once every 8 minutes, once every 9 minutes, once every 10 minutes, once every 11 minutes, once every 12 minutes, once every 13 minutes, once every 14 minutes, once every 15 minutes, once every 16 minutes, once every 17 minutes, once every 18 minutes, once every 19 minutes, once every 20 minutes, once every 21 minutes, once every 22 minutes, once every 23 minutes, once every 24 minutes, once every 25 minutes, once every 26 minutes, once every 27 minutes, once every 28 minutes, once every 29 minutes, once every 30 minutes, once every 31 minutes, once every 32 minutes, once every 33 minutes, once every 34 minutes, once every 35 minutes, once every 36 minutes, once every 37 minutes, once every 38 minutes, once every 39 minutes, once every 40 minutes, once every 41 minutes, once every 42 minutes, once every 43 minutes, once every 44 minutes, once every 45 minutes, once every 46 minutes, once every 47 minutes, once every 48 minutes, once every 49 minutes, once every 50 minutes, once every 51 minutes, once every 52 minutes, once every 53 minutes, once every 54 minutes, once every 55 minutes, once every 56 minutes, once every 57 minutes, once every 58 minutes, once every 59 minutes, and once every 60 minutes.

In certain embodiments, the frequency and/or schedule are selected from approximately once every hour, once every 2 hours, once every 3 hours, once every 4 hours, once every 5 hours, once every 6 hours, once every 7 hours, once every 8 hours, once every 9 hours, once every 10 hours, once every 11 hours, once every 12 hours, once every 13 hours, once every 14 hours, once every 15 hours, once every 16 hours, once every 17 hours, once every 18 hours, once every 19 hours, once every 20 hours, once every 21 hours, once every 22 hours, once every 23 hours, once every 24 hours, once every 25 hours, once every 26 hours, once every 27 hours, once every 28 hours, once every 29 hours, once every 30 hours, once every 31 hours, once every 32 hours, once every 33 hours, once every 34 hours, once every 35 hours, once every 36 hours, once every 37 hours, once every 38 hours, once every 39 hours, once every 40 hours, once every 41 hours, once every 42 hours, once every 43 hours, once every 44 hours, once every 45 hours, once every 46 hours, once every 47 hours, and once every 48 hours.

In certain aspects, the present disclosure provides a wearable printed circuit board (PCB) comprising: a multi-layer and/or flexible substrate carrying a microcontroller unit (MCU), a power regulation stage with a DC-DC converter supplying 1-24 V for stimulation pulses, one or more output drivers for pulse generation, and/or a Bluetooth Low Energy (BLE) radio transceiver or equivalent wireless module for programming and data logging; wherein: the output driver(s) is/are selected from transistor(s), MOSFET(s), and power switch(es); and the wearable printed circuit board is configured to measure signals from at least one sensor, process the signals using the integrated microcontroller unit and deliver an electrical stimulation pulse upon exceeding a predetermined threshold.

In certain embodiments, the device or wearable PCB further comprise harvesting pads and/or microneedles placed on the skin, wherein the wearable PCB is connected to the harvesting pads and/or microneedles placed on the skin, and the wearable PCB produces an emitted pulse.

In certain embodiments, the device or wearable PCB further comprise microneedles and/or electrode pads, wherein the microneedles and/or electrode pads are electrically coupled to output and ground pins, respectively, of the wearable PCB, and the microneedles and/or electrode pads deliver an emitted pulse.

In certain embodiments, the wearable PCB further comprises a microneedle connection, wherein the microneedle connection is a microneedle array or single microneedle; wherein (1) each microneedle array or single microneedle is soldered or mechanically attached to a dedicated copper pad; (2) a ground return path is routed through a dedicated ground pad, ensuring minimal cross-talk with sensor traces; (3) the microneedle array is encapsulated or coated except at its tip, so that only the tip contacts the subdermal environment, reducing risk of short circuits or patient discomfort; and/or (4) high-voltage traces to the microneedle pad include protective coatings (e.g., silicone encapsulant or parylene) to maintain electrical isolation from other on-board circuits.

In certain embodiments, the wearable PCB further comprises a connected sensor, wherein the connected sensor gives a signal that enables the wearable PCB to emit a pulse. In further embodiments, the sensor is a strain sensor. In yet further embodiments, the strain sensor is made from laser induced graphene.

In certain embodiments, the sensor comprises: a gauge factor in the range of 5-10, enabling detection of low (<5%) strain events; a flexible substrate (SEBS or comparable elastomer) with thickness of 75 μm; a patterned graphene trace formed by applying XW laser power on polyimide, subsequently transferred onto the SEBS substrate; and electrical interconnects via serpentine copper traces to reduce mechanical stress and provide robust bonding to the wearable PCB.

In certain embodiments, the sensor is selected from a pressure sensor, a chemical sensor, or an electrical sensor.

In certain embodiments, the device or wearable PCB further comprise a microcontroller unit (MCU), wherein the serpentine copper traces are used to connect the MCU to other components on the wearable PCB.

In certain embodiments, the wearable PCB comprises electrical interconnects via serpentine copper traces for signal and power routing, and wherein each serpentine copper trace: 1) follows a repeated signal path with amplitude and pitch between 0.5-15 mm, providing mechanical stretchability and bending tolerance; 2) is fabricated by laser micromachining of laminated Cu on polyimide or an elastomer substrate; and/or 3) maintains conductive integrity under tensile strains up to 10%, reducing mechanical failure when the wearable PCB bends to conform to the skin.

In certain embodiments, the device or wearable PCB further comprise an implantable printed circuit board (PCB) inside of the body, wherein the implantable PCB emits a pulse and the implantable PCB is implanted, ingested or inserted.

In certain embodiments, the emitted pulse comes from the gastrointestinal (GI) tract, the subcutaneous space, the intraperitoneal space, the nasal cavity, the epidermal space, the subdermal space, the intrathoracic space, or a space within an organ or on the wall of an organ; wherein the implantable PCB is implanted within the organ or on the wall of the organ.

In certain embodiments, the device or wearable PCB further comprise harvesting pads, wherein the harvesting pads are placed inside of the body on an implantable printed circuit board (PCB), wherein the harvesting pads are implanted, ingested, or inserted in the body.

In certain embodiments, the device or wearable PCB further comprise an implantable printed circuit board (PCB) inside of the body, wherein the implantable PCB comprises: a battery or energy-harvesting system providing long-term power; a microcontroller unit (MCU) or transistor-based logic for interpreting the received voltage gradient from subdermal or intrabody electrodes; a micro-scale stimulator output triggered upon detection of a threshold gate voltage; harvesting pads spaced ≥5 mm apart on the PCB for capturing differential signals in tissue; wherein: the battery comprises 1.55 V silver oxide coin cell; the energy-harvesting system provides long-term power up to over 300 days; and the micro-scale stimulator output is selected from nerve cuff or LED.

In certain embodiments, the theoretical maximum printed circuit board dimensions size in typical rodent or small-animal models is 30 mm ×20 mm for practical surgical implantation; maximum size in human-scale embodiments is 50 mm×50 mm, wherein the maximum size is constrained by the intended anatomical site; and/or the theoretical minimum size can be as small as 5 mm×5 mm given miniaturized components and micro-batteries, suitable for certain deep tissue implantations.

In certain embodiments, the harvesting pads are located in the gastrointestinal (GI) tract, the subcutaneous space, the intraperitoneal space, the nasal cavity, the epidermal space, the subdermal space, the intrathoracic space, or a space within an organ or on the wall of an organ; wherein the implantable PCB is implanted within the organ or on the wall of the organ.

In certain embodiments, the harvesting pads are located in different organs in the body.

In certain embodiments, the harvesting pads are wearable.

In certain embodiments, the harvesting pads are implantable, and the transistor of the wearable and/or implantable printed circuit board measures no greater than 158 mm² in surface area.

In certain embodiments, the device or wearable PCB further comprise an in-body communication pathway (IBC) wherein the in-body communication (IBC) pathway extends battery life to 300 days or more to the receiving device by requiring 1 μA idle current draw, improving device lifetime in comparison to equivalent devices using Bluetooth or NFC protocol.

In certain embodiments, the in-body communication pathway improves the battery life of receiving devices compared to devices that use Bluetooth or NFC.

In certain embodiments, the in-body communication pathway improves the battery life of emitting devices compared to devices using Bluetooth or NFC.

In certain aspects, the present disclosure provides methods of treating, preventing, or diagnosing a disease or disorder, comprising attaching to a subject in need thereof a device or wearable PCB of the disclosure.

This disclosure describes an implementation of in-body communication where a wearable PCB reads out a sensor reading, chooses when to deliver a voltage pulse into the tissue, delivers the voltage pulse into the tissue, and an implantable PCB reads out that voltage pulse. By removing the energy intensive communication and decision-making protocols from the implantable device, the device can be much smaller and also last much longer compared to when utilizing other communication methods. We demonstrate that this system can trigger multiple devices in vivo using an example of strain sensors triggering sciatic nerve stimulation and leg movement.

Safety: To create a safe communication system, it is necessary to have the following: (1) a limit on the amount of current that passes through the body [currents higher than 5 mA will cause damage, and we provide histology demonstrating that there are current values that do not stimulate high levels of scar tissue formation]. (2) Microneedles are minimally invasive, and the placement of the microneedles in the skin for extended periods of time could cause fibrosis and reduce efficacy or create an infection [there is precedent to having devices inside of the subcutaneous space for up to 10 days in the continuous glucose monitor]. (3) the materials in the body must be biocompatible [the only materials that are required to be in direct contact with the body are the injection probes and the harvesting pads, and these can be made from any biocompatible conductor or semiconductor. (4) The speed at which the implantable reacts to the signal is critical to maintaining precision control. This means both the time that it takes to turn on and the time that it remains on after the signal is sent.

### EXAMPLES

### Example 1: Wearable Printed Circuit Board for Delivering Electrical Stimulation Pulses Into the Tissue, Reading out a Sensor Measurement, and Communicating With a Computer and the Accompanying Implantable Circuit

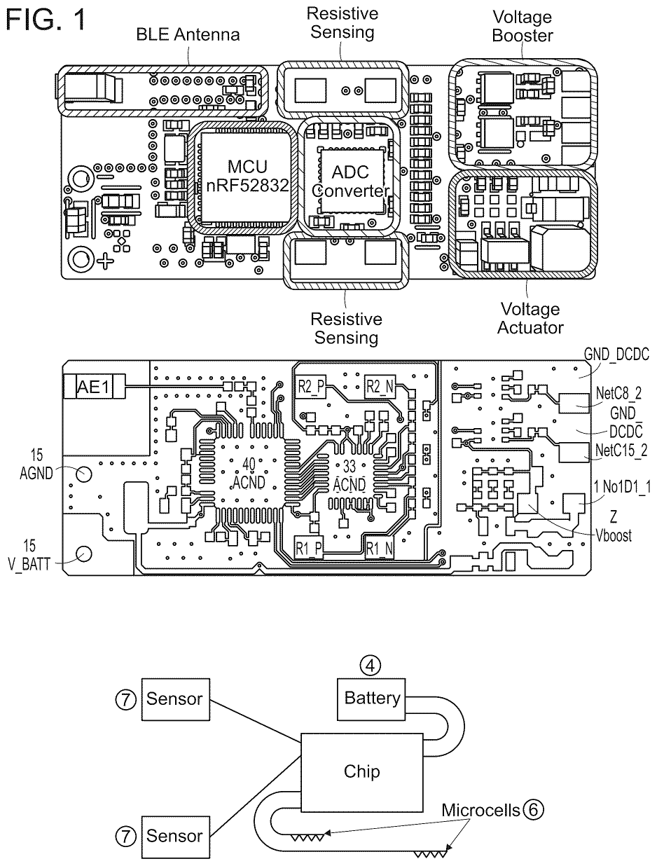

FIG. 1 describes an exemplary architecture and component of the wearable PCB layout and its major functional blocks, illustrating how each component is arranged for optimal signal generation, sensing, and wireless communication. The components of Wearable Printed Circuit Board are listed below:

### 1. Microcontroller Unit (MCU):

An nRF52832 system-on-chip placed at the center of the PCB processes executing the main control algorithm, reading sensor data, and driving the stimulation pulses.
The system is designed with minimal traces length and physical hardware for data communication, operation and critical signals.

### 2. Analog-to-Digital Converter (ADC):

ADS 1292 or equivalent 24-bit ADC, placed adjacent to the MCU to minimize noise on analog front-end signals coming from strain sensors or other biosensors.
Shielded and/or separated by ground planes to reduce electromagnetic interference and/or logic interference and/or control interference.

### 3. Power Management Block:

ADC-DC boost converter that steps up the LiPo battery voltage (3-4V nominal) to the custom/required/range of stimulation voltage (1-24 V adjustable).
An on-board linear regulator and/or low-dropout (LDO) regulator for powering analog circuits (around 1.8-3.3 V)

### 4. Battery and Charging Section:

A 3.7 V LiPo battery with magnetic charging or standard JST connector.
Placed away and/or separated from analog sensing traces to reduce noise coupling.

### 5. Stimulation Output Stage:

A set of MOSFET/transistor/Power Switch drivers that generate user-programmable monophasic or biphasic pulses.
High-voltage lines are routed at the PCB perimeter, for a robust clearance from low-voltage digital signals.

### 6. Microneedle/Electrode Connectors:

One/Two or more output pads with mechanical anchor points for microneedles.
Ground/Return reference is routed as a dedicated plane and/or area and/or wide trace to reduce resistive losses.

### 7. Sensor Input Ports (Serpentine Traces):

Flexible serpentine leads that interface with the LIG sensor or alternative strain sensor and/or biosensor.
Designed to maintain mechanical compliance and reduce stress at solder/joints.

### 8. Continuous Analog Sensor Collection

The strain or bio-signal continuously measured by a Wheatstone-type resistor network of resistor branches series with adjustable reference, thereby allows differential resistance sensing, and fine-tuning of offset and gain for further calibration, calibration curve, and measurement precision.

### 9. Wireless Connectivity (BLE Antenna):

An impedance matched/optimized 2.4 GHz antenna area), properly cleared of ground copper and large components for optimal BLE performance.

### PCB Design Rational

The wearable circuit board is designed for compact layout to ensure wearability, with the MCU and ADC placed near each other to reduce inductances on sensor lines and improve analog performance. Ground planes and careful component placement minimize EMI for the 24-bit signals, and DC boosting, thereby achieving low-noise sensing and stimulation. Flexible serpentine copper traces provide mechanical compliance, allowing the board to conform to surface while accommodating strain relief during movement. A configurable DC-DC boost converter delivers stimulation voltages from 1 V up to 24 V, accommodating diverse pulse requirements. Finally, microneedle integration lowers tissue-electrode impedance for robust subdermal voltage injection, enabling reliable in-body communication.

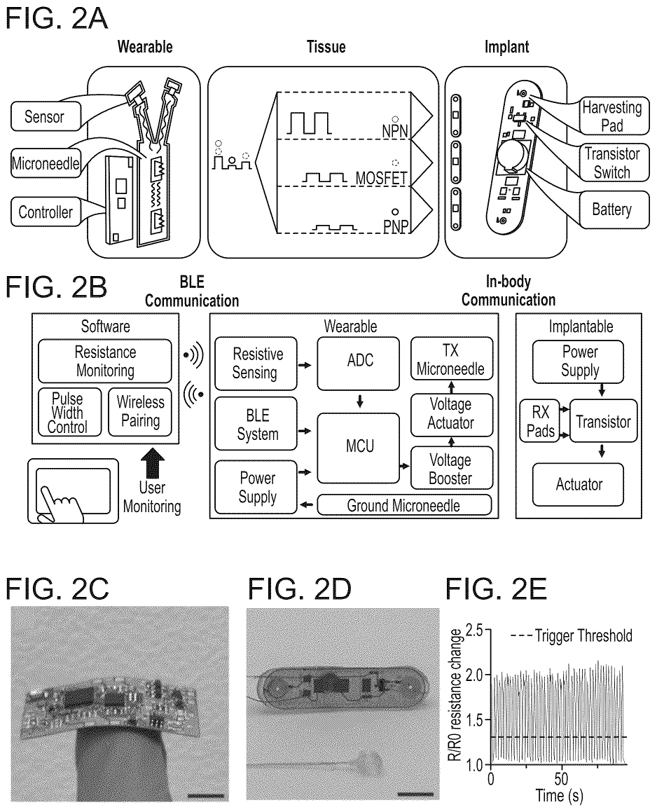

The in vivo SWANS system utilizes a custom-designed wearable printed circuit board (PCB) containing interchangeable physical or chemical sensors, a control unit, and a signal generator to execute a closed-loop algorithm that injects electrical pulses through body tissue to implantable therapeutic actuators when the sensor readouts reach given thresholds (FIG. 2A).

FIG. 2B provides a block diagram of the wearable (FIG. 2C) and implantable (FIG. 2E) modules. The wearable device can receive input from multiple sensors at once as well as deliver electrical pulses with varying characteristics that uniquely trigger the different transistor switches found in implants throughout the body. In our proof-of-concept example, we utilize two highly sensitive laser-induced graphene-based strain sensors to uniquely send signals to sciatic nerve stimulators on each hind leg when surpassing a given threshold readout (FIG. 2F). These sensors are capable of large resistance changes [(R/Rₒ=2) at strains (<5%)] allowing for detection of small movements such as forelimb motion in a small animal or human finger flexion. The sensors are connected to the wearable PCB via serpentine traces. An analog-to-digital converter (ADC) and microcontroller unit (MCU) process measured strain data in real time which is stored by a BLE-enabled user interface. When a threshold value is reached on the sensor, the wearable PCB delivers an electrical pulse into the tissue via subdermal microneedle probes connected to the PCB via serpentine traces. The implantable devices detect generated voltage gradients through two RX pads and actuate according to physical signals detected by the wearable module.

### Serpentine Traces

### Fabrication of Bi-Layer Copper Flexible 2CH Interconnect

The synthesis of polydimethylsiloxane (PDMS) was conducted using Sylgard 184 (Dow) with a 10:1 PDMS to crosslinker ratio. The prepared PDMS was subsequently spin-coated onto a stainless-steel substrate to ensure a smooth surface, set at a speed of 1000 rpm for a duration of 35 seconds, with a ramp period of 5 seconds included. The elastomer was then cured at 120 °C on a hotplate for 30 minutes. A copper (Cu) foil was laminated onto the PDMS surface; due to the inherent Van der Waals forces, no additional adhesive was required to achieve adhesion, attributed to the high adhesion energy of PDMS molecules that promote stable interfacial contact.

Following the initial lamination, a layer of low-modulus silicone elastomer with high surface energy (Ecoflex-GEL, Smooth-On Inc.) was applied on the laminated Cu film via spin coating at 1500 rpm for 35 seconds. The applied elastomer was then cured at 65 °C for 1 hour, providing a uniform insulating layer. Subsequently, a second Cu foil was laminated onto the cured elastomer layer, resulting in a bi-layer configuration of parallel Cu films separated by a thin elastomer layer that serves as an insulator to prevent electrical shorting.

To pattern the Cu-elastomer-Cu composite, laser micromachining was performed using an OPTEC Femtosecond Laser system. A multi-step cutting process was employed to minimize substrate damage when processing the relatively thick composite layer. Using the top Cu film as the baseline focal point, patterning was conducted at 65% laser power for five repetitions. The focal point was then lowered by 250 μm, and the cutting process was repeated. This sequence was iterated three times to ensure full penetration through the composite to the bottom Cu layer. After laser processing, negative material was carefully removed, and the resulting bi-layer interconnect was laminated with PVA water-soluble tape (5414 Water-Soluble Wave Solder Tape, 3M) to facilitate its transfer from the PDMS surface for further integration onto fabric adhesive substrates.

### Fabrication of PI/Cu Flexible Interconnect

The PDMS precursor was prepared with Sylgard 184 (Dow) at a 10:1 PDMS to crosslinker ratio and then spin-coated onto a stainless-steel substrate at 1000 rpm for 35 seconds, including a 5-second ramp, to achieve a smooth elastomer layer. Curing was conducted on a hotplate at 120 °C for 30 minutes. After curing, Cu foil was laminated onto the PDMS surface, where it adhered naturally due to Van der Waals forces, facilitated by the high adhesion energy of PDMS molecules.

Following Cu lamination, a heat-activated polyimide (PI) adhesive film (Pyralux LF, DuPont) was applied onto the Cu foil, and bonding between the Cu and PI layer was achieved by heat pressing at 400 °C for 15 seconds. The thermoplastic adhesive was activated under heat, forming a cross-linked polymer matrix between the PI and Cu layers, thereby creating a permanent bond.

The PI/Cu composite film was subsequently patterned using laser micromachining with an OPTEC Femtosecond Laser at 75% power, applied in five repetitions. The laser cutting parameters were meticulously optimized to remove material effectively while preventing thermal damage, which could introduce carbonized impurities along the patterned edges. Upon completion of the laser patterning, the negative material was removed, and the remaining bi-layer interconnect was laminated with PVA water-soluble tape (5414 Water-Soluble Wave Solder Tape, 3M) to facilitate detachment from the PDMS substrate and subsequent transfer to a fabric adhesive for final mounting.

Wearable package: A wearable pulse generation system was developed, featuring a flexible PCB (fPCB) circuit layered with polyimide-copper for skin conformity and flexibility.

The wearable device connects with an Android application, allowing contactless control of pulse delivery and custom adjustments of parameters such as frequency (0.01-1 Hz) and pulse width (1-1000 ms). The fPCB utilizes the nRF52832 microcontroller (MCU) architecture, selected for its industry standard low-power Bluetooth capabilities. This wireless system integration allows user-defined control via the mobile app for open/close/remote controls.

The device's voltage range spans from 1V to 12V, powered by a custom-configured, controllable DC-DC step-up voltage regulator and booster. The design is customizable with stimulation scenarios using both transistors and MOSFETs, regardless of NPN or PNP types, allowing for programmable voltage amplitude, pulse width, and frequency to generate dynamic pulses. A monophasic square-wave pulse can be generated through firmware control of the microcontroller's on-board pull-up resistor, making the device fit for various stimulation protocols.

To facilitate input-triggered reading/actuation, an ADS 1292 24-bit analog-to-digital converter (ADC) is integrated, interfacing with resistive sensing elements. These elements detect mechanical stimulation, such as strain changes, and can initiate pulse generation in response to physical cues and for biofeedback system.

The wearable is powered by a 3.7V LiPo battery, equipped with a magnetic charging circuit to enable standalone operation, eliminating the need for an external power supply. The Android or iOS app further supports real-time data monitoring and control, creating a comprehensive interface for remote access, wireless connectivity, and actuation commands.

Implantable package: The implantable device, measuring 25 by 10 mm, is designed to function as a transdermal signal harvester and programmable output stimulator. The device is powered by a 1.55V silver oxide battery, carefully selected for its compatibility with gate voltage requirements and an operational lifetime extending to approximately 500 days. The entire implantable fPCB is encapsulated in PDMS (Sylgard 184, Dow) using a 2-piece mold that ensures that gold contact pads for signal harvesting interface with surrounding tissue.

### Example 2: Ultra-Sensitive Laser-Induced Graphene Sensor for Movement Detection

In closed-loop control, actuation only occurs in response to a sensed element. In this closed-loop, a laser-induced graphene (LIG) sensor is affixed to the skin of the small animal using brown medical tape. The laser-induced graphene sensor is fabricated by using a conventional carbon dioxide laser on a polyimide substrate to induce graphene on the surface of the substrate. The graphene is then transferred to a flexible substrate, such as styrene-ethylene-butylene-styrene (SEBS) copolymer, by repeatedly applying 100N of force onto the polyimide while the SEBS substrate is underneath it. After removing the polyimide sheet, a thin layer of graphene remains on the SEBS substrate.

The laser-induced graphene (LIG) strain sensor displays a gauge factor of 5-10 demonstrates large relative resistance changes with only small mechanical deformations (<5% strain).

In practical terms, these sensors can detect subtle limb or finger movements down to sub-millimeter displacements. The minimum resolvable strain can be as low as 0.5% in the presence of environmental noise.

### Sebs-Based Serpentine and LIG Sensor Fabrication

First, a PI/Cu composite film is laminated onto a PDMS-coated aluminum (Al) plate. The PDMS layer is spin-coated at 500 rpm with a 5-second ramp and a 30-second spin duration before curing-for a smooth bonding interface. A layer of Pyralux® PI is then heat-pressed onto the Cu film at 370 °C for 10 seconds to form a robust bond. Next, serpentine circuit traces are laser-machined using an Optec femtosecond laser at 50% power with 5 repetitions to cleanly define the pattern.

Meanwhile, 50 mg/ml SEBS is dissolved in cyclohexane and drop-casted onto an coated Al plate, followed by curing at 25 °C for 180 minutes. The patterned PI/Cu serpentine is transferred to this SEBS film. In parallel, laser-induced graphene (LIG) is generated on a separate polyimide sheet via CO₂ laser at 5% power. The resulting LIG layer is “stamped” (10 passes under a 100N of force) onto the exposed Cu pads of the serpentine trace, thereby integrating the LIG sensing region. A second SEBS layer is overlaid, and the natural SEBS-SEBS adhesion provides a reliable seal around the LIG. Finally, the entire assembly is peeled from the Al plate and transferred onto a brown tape substrate for final use as a flexible, skin-mountable sensing/stimulation patch.

LIG strain sensor: Polyimide (Grainger) was laser-cut with a Trotec Speedy laser (5% power, 0.2 speed, 16 kHz) to produce conductive graphene traces that were subsequently stamped onto an elastomer. 0.5 g of styrene-ethylene-butylene-styrene (SEBS; Asahi Kasei) was dissolved in 10 mL cyclohexane at 70 °C and drop-casted onto a glass slide. The SEBS film was then lifted and used for LIG stamping.

### Fabrication of Ultrathin Uniaxial Laser-Induced Graphene (LIG) Sensor

The fabrication of the uniaxial LIG sensor began with the synthesis of styrene-ethylene-butylene-styrene (SEBS) in a dissolution concentration at X %, homogenized on a hotplate at a determined temperature for a precise duration to ensure uniform solution characteristics. The stainless steel (SS) substrate was prepared by spraying it with an anti-stick aerosol (Ease Release 200, Reynolds Advanced Materials) to ease subsequent material removal. SEBS solution was then drop-cast onto the SS substrate and cured at 35 °C for 2 hours, resulting in a uniform SEBS film. Laser micromachining using the OPTEC Femtosecond Laser was employed to achieve precise film patterning, after which the negative material was carefully removed.

For the LIG component, a polyimide (PI) sheet of 75 um thickness underwent CO₂ laser cutting at 5% power, which induced the formation of a laser-induced-graphene layer on its surface. The LIG strip was transferred onto the stretchable SEBS substrate and Cu conductive layer through mechanical stamping, with pressure applied at a predetermined force over ten repetitions to ensure strong adhesion. The interconnect and LIG components were then sealed together by SEBS-SEBS film adhesion, which relies on Van der Waals forces for a secure bond, achieving both durability and stretchability without increasing overall thickness. Finally, the exposed copper pads of the PI/Cu interconnect were soldered to the 2CH lead from the device, and the entire assembly was bonded to a fabric adhesive for secure mounting onto biological substrates.

### LIG Strain Sensor Benchtop Characterization

To measure the resistance during stretching, we attached the samples to a homemade stretching station and connected the samples to a tabletop multimeter. A custom Lab View program simultaneously collected force, displacement, and resistance data. Samples were stretched at 1 mm/s up to 20% strain for up to 50 cycles.

### Example 3: Method for Actuating Multiple Body-Internalized Devices Upon Signal Transmission

The wearable PCB can contain multiple output pins that each deliver a different voltage pulse magnitude to the microneedles. This creates a different voltage gradient in the body which can in turn trigger different transistor gate voltages in the implants.

### Example 4: Method for Actuating a Device Upon Signal Transmission Upon Detecting a Signal From a Disease Morbidity

Signal transmission (the delivery of an electrical pulse into body tissue) can occur due to user input (e.g., user inputs command onto smart phone which relays data to the wearable, thereby actuating a device). Signal transmission can occur on a preprogrammed frequency or schedule such as once a second, once every minute, once every hour, once every day, once every month, etc. Signal transmission can also be tied to a sensor such as a body-worn strain sensor or some other physical sensor such that when the sensor hits a certain threshold that a signal is sent.

A chemical sensor such as a continuous glucose monitor, or a ketone monitor could also be used. Once a threshold value is crossed with regards to the sensing objective, signal transmission could automatically happen from a printed circuit board. Additionally, a control algorithm (such as a proportional, integral, and derivative (PID) controller, model predictive control (MPC) controller, or a fuzzy logic control (FLC) controller), could control when signal transmission occurs so that actuation occurs in advance of a signal deviation (such as minutes before a heart arrythmia or a hypoglycemic episode).

### Example 5: Alternative Devices to Be Utilized With Signal Transmission

The transistor circuit can be integrated with a number of electromechanical devices. We have demonstrated use in light emitting diodes (LEDs), nerve cuffs, pumps, and triggerable auto-injectors. It can also be used in other electrical stimulators such as gastric stimulators or pacemakers. It can also be used with other electromechanical devices such as motors or solenoids. It can also be used with sensors. It can also be used with microcontrollers that have dedicated BLE modules.

### Closed-Loop Demonstration via In-Body Communication

Subjects were shaven and prepared for aseptic surgery using 3 cycles of chlorohexidine and 70% ethanol application. Female Sprague Dawley rats (200-300 g, Charles River) were anesthetized with 2% isoflurane in balanced oxygen. Under sterile conditions with external body warming, a 2-cm incision was made on the left thigh. About 1 cm of the sciatic nerve proximal to the tibial and peroneal bifurcation was inserted into the nerve cuff electrode (World Precision Instruments). Nerve cuff leads were tunneled through the muscle layer (closed with 4-0 silk sutures (Ethicon)) into the subcutaneous space where the implantable FPCB was placed. The skin was secured with Michel clips.

The subject was placed in a prone position and was outfitted with the wearable FPCB including microneedles and a soft strain sensor. A soft tether was applied to each of the forelimbs and the tether was controlled by the researcher to simulate extension of the forelimb and a natural walking motion. Electrical characterization of the wearable and implantable FPCBs was completed using an oscilloscope. Resistance measurements were collected using a tabletop multimeter (Keithley). Frames from video recordings set up for monitoring each of the hindlegs were extracted and used to determine the degree of limb flexion.

The wearable and implantable devices operate in a closed-loop configuration for adaptive therapeutic response. The wearable device continuously monitors motion through a Laser-Induced Graphene (LIG) sensor, capturing motion range and detecting boundary conditions. Upon detecting a boundary condition change, the wearable's pulse generator activates a voltage boost, sending an electrical trigger to the implantable device.

This closed-loop control enables targeted neurostimulation via a microneedle interface in the implantable system, precisely adjusting the stimulation intensity in response to user movement or external stimuli. The implantable device, acting as an electrical gate, modulates the stimulus through programmable parameters, ensuring effective and responsive therapy delivery within the in vivo model.

### INCORPORATION BY REFERENCE

All U.S. and PCT patent application publications and U.S. patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1-35. (canceled)

36. A printed circuit board (PCB) comprising:
a multi-layer and/or flexible substrate carrying a microcontroller unit (MCU),
a power regulation stage with a DC-DC converter supplying 1-24 V for stimulation pulses, and
one or more output drivers for pulse generation,
wherein:
the output driver(s) is/are selected from transistor(s), MOSFET(s), and power switch(es); and
the printed circuit board is configured to measure signals from at least one sensor, process the signals using the integrated microcontroller unit and deliver an electrical stimulation pulse upon exceeding a predetermined threshold.

37. The PCB of claim 36, wherein the PCB is wearable.

38. The PCB of claim 36, wherein the PCB comprises a Bluetooth Low Energy (BLE) radio transceiver or equivalent wireless module for programming and data logging.

39. The PCB of claim 36, further comprising harvesting pads and/or microneedles placed on the skin, wherein the PCB is connected to the harvesting pads and/or microneedles placed on the skin, and the PCB produces an emitted pulse.

40. The PCB of claim 36, further comprising microneedles and/or electrode pads, wherein the microneedles and/or electrode pads are electrically coupled to output and ground pins, respectively, of the PCB, and the microneedles and/or electrode pads deliver an emitted pulse.

41. The PCB of claim 36, further comprising a microneedle connection, wherein:
the microneedle connection is a microneedle array or single microneedle;
each microneedle array or single microneedle is soldered or mechanically attached to a dedicated copper pad;
a ground return path is routed through a dedicated ground pad;
the microneedle array is encapsulated or coated except at its tip; and
high-voltage traces to the microneedle pad include protective coatings.

42. The PCB of claim 36, further comprising a connected sensor, wherein the connected sensor gives a signal that enables the PCB to emit a pulse.

43. The PCB of claim 42, wherein the sensor is a strain sensor.

44. The PCB of claim 43, wherein the strain sensor is made from laser induced graphene.

45. The PCB of claim 44, wherein the sensor comprises:
a gauge factor in the range of 5-10;
a flexible substrate (SEBS or comparable elastomer) with a thickness of about 75 μm;
a patterned graphene trace formed by applying laser power on polyimide, subsequently transferred onto the SEBS substrate; and
electrical interconnects comprising serpentine copper traces.

46. The PCB of claim 42, wherein the sensor is selected from a pressure sensor, a chemical sensor, and an electrical sensor.

47. The PCB of claim 45, wherein the serpentine copper traces connect the MCU to other components on the PCB.

48. The PCB of claim 45, wherein each serpentine copper trace:
follows a repeated signal path with amplitude and pitch between 0.5-15 mm;
is fabricated by laser micromachining of laminated Cu on polyimide or an elastomer substrate; and
maintains conductive integrity under tensile strains up to 10%.

49. An implantable printed circuit board (PCB), wherein the implantable PCB comprises:
a battery or energy-harvesting system;
a microcontroller unit (MCU) or a transistor(s) for interpreting a received voltage gradient from subdermal or intrabody electrodes;
a micro-scale stimulator output triggered upon detection of a threshold gate voltage; and
harvesting pads spaced at least 5 mm apart on the implantable PCB; wherein:
the battery comprises a 1.55 V silver oxide coin cell; and
the micro-scale stimulator output is a nerve cuff or an LED.

50. The implantable PCB of claim 49, wherein:
the implantable PCB has a first dimension and a second dimension;
the first dimension is about 5 mm to about 50 mm; and
the second dimension is about 5 mm to about 50 mm.

51. The implantable PCB of claim 49, wherein the harvesting pads are located in the gastrointestinal (GI) tract, the subcutaneous space, the intraperitoneal space, the nasal cavity, the epidermal space, the subdermal space, the intrathoracic space, or a space within an organ or on the wall of an organ; and the implantable PCB is implanted within the organ or on the wall of the organ.

52. The implantable PCB of claim 49, wherein the harvesting pads are wearable.

53. The implantable PCB of claim 49 comprising a transistor(s), wherein the transistor(s) measures no greater than about 158 mm² in surface area.

54. The implantable PCB of claim 49, wherein the battery has a battery life of about 300 days or more.

55. A method of treating, preventing, or diagnosing a disease or disorder, comprising attaching to a subject in need thereof a PCB of claim 36.
